(19) United States
(12) Patent Application Publication
(10) Pub. No.: US 2025/0037898 A1
(43) Pub. Date: Jan. 30, 2025

(54) COATED ELECTRICAL CONDUCTOR AND METHOD FOR MANUFACTURING SAME

(71) Applicant: Heraeus Medevio GmbH & Co. KG, Hanau (DE)

(72) Inventors: Suman NARAYAN, Hanau (DE); Katharina MUSIOL, Hanau (DE)

(21) Appl. No.: 18/779,603

(22) Filed: Jul. 22, 2024

(30) Foreign Application Priority Data
Jul. 24, 2023 (DE) 102023119482.8

(51) Int. Cl. H01B 1/02; A61N 1/04; C22C 5/04; C25D 7/00; C25D 9/04; H01B 1/12; H01B 5/00; H01B 13/00

(52) U.S. Cl. CPC H01B 1/02; C22C 5/04; C25D 7/00; C25D 9/04; H01B 1/124; H01B 5/00; H01B 13/0036; A61N 1/04

(57) ABSTRACT

An electrical conductor, in particular a medical electrode, comprising a substrate having a surface on which is arranged a first layer that comprises a platinum-iridium alloy and a second layer that comprises a conductive polymer and is arranged on the first layer. The invention further relates to a method for manufacturing such an electrical conductor, in particular for manufacturing a medical electrode.

### CROSS-REFERENCE TO RELATED APPLICATIONS

This application claims priority pursuant to 35 U.S.C. 119(a) to German Application No. 102023119482.8, filed Jul. 24, 2023, which application is incorporated herein by reference in its entirety.

### FIELD OF THE INVENTION

The invention relates to an electrical conductor, in particular a medical electrode, comprising a substrate having a surface on which is arranged a first layer that comprises a platinum-iridium alloy and a second layer that comprises a conductive polymer and is arranged on the first layer. The invention further relates to a method for manufacturing such an electrical conductor, in particular for manufacturing a medical electrode.

### BACKGROUND OF THE INVENTION

Medical devices, and in particular active implantable medical devices, generally comprise electrodes for electrically stimulating body tissue or for measuring electrical signals generated by this. Examples of body tissues include muscles and nerves. These electrodes also include electrical conductors.

There are a number of important requirements applicable to the aforementioned electrical conductors. A first requirement is that the electrical conductor should have a very low impedance, in particular at the lower frequencies in the range of 0.1 Hz to 100 Hz. This low frequency range is of particular importance for the medical devices mentioned above. A very low impedance is essential to ensure a good signal-to-noise ratio. A further requirement is that the electrical conductor should have a high charge storage capacity. A further requirement is that the electrical conductor should have long-term stability. This means that the electrical properties, for example impedance and charge storage capacity, of the electrical conductor should substantially not change over time. This is important for medical devices used for stimulation, since the electrical signal should not change over time. Such long-term stability is also important for medical devices used for mapping and recording, since it affects the measurement accuracy of the medical device.

Electrical conductors very often comprise a substrate, but not a coating layer. A lack of a coating layer has the disadvantage that such electrical conductors have a very high electrical impedance, in particular at lower frequencies in the range from 0.1 Hz to 100 Hz. Uncoated electrical conductors also have a low charge storage capacity and low signal-to-noise ratio.

For example, it is known from U.S. Pat. No. 10,219,715 B2 that the electrical properties of the electrical conductor, for example impedance and charge storage capacity, can be improved by using laser ablation to structure the substrate.

However, even in this case the impedance remains too high, in particular in the range from 0.1 Hz to 100 Hz, and the charge storage capacity too low.

The substrate can be coated with an electrically conductive polymer in order to improve the aforementioned electrical properties, as disclosed in, for example, US 2019/0159833 A1. However, adhesion of the electrically conductive polymer to the substrate is difficult to achieve, in particular if the electrical conductor is subjected to mechanical stresses and forces. As a result, it very often occurs that sections of the electrically conductive polymer detach from the substrate. The adhesion of a polymer coating can be improved if the substrate is laser-structured, as disclosed in, for example, EP 3500419 B 1. Yet, even in this case, adhesion remains problematic. This lack of adhesion not only affects the patient but also the electrical properties and long-term stability of the electrical conductor.

To improve adhesion of the electrically conductive polymer to the substrate, the substrate can comprise a metal, such as iron, silver, copper, nickel, palladium, platinum, gold, iridium, titanium, hafnium, niobium, tantalum, cobalt, chromium, zirconium, rhenium, tungsten, molybdenum, or an alloy, such as a platinum alloy, such as a platinum-iridium alloy, or steel. This can improve adhesion between the substrate and conductive polymer, as described, for example, in U.S. Pat. No. 11,495,369 B2.

In the paper entitled “Long-Term Stable Adhesion for Conducting Polymers in Biomedical Applications: IrOx and Nanostructured Platinum Solve the Chronic Challenge,” ACS Appl. Mater. Interfaces 2017, 9, 189-197 by Boehler et al., the bonding of conducting polymers, such as poly(3,4-ethylenedioxythiophene) (PEDOT), to iridium oxide is described. However, such a bonding to iridium in the form of an alloy is denied.

Significant efforts continue to be made to provide electrical conductors, in particular medical electrodes, that have low impedance while maintaining high mechanical strength.

### Objects

One object of the present invention is to overcome, at least in part, one or more of the disadvantages resulting from the prior art.

It is a further object of the invention to provide an electrical conductor that has reduced impedance.

It is a further object of the invention to provide an electrical conductor that has an increased charge storage capacity.

It is a further object of the invention to provide an electrical conductor that has increased robustness to damage resulting from mechanical forces and stresses.

Examples of damage resulting from mechanical forces include damage that occurs when, for example, the electrical conductor is inserted into or withdrawn from the body of a mammal or handled by an operator.

It is a further object of the invention to provide an electrical conductor that has improved adhesion of a conductive polymer to its surface.

It is a further object of the invention to provide an electrical conductor that has improved long-term stability.

It is a further object of the invention to provide a method for manufacturing an electrical conductor by means of which at least some of the objects already described are at least partially solved.

### PREFERRED EMBODIMENTS OF THE INVENTION

A contribution to the at least partial fulfillment of at least one of the aforementioned objects is made by the features of the independent claims. The dependent claims provide preferred embodiments that contribute to at least partial fulfillment of at least one of the objects.

A first embodiment of the invention is an electrical conductor, in particular a medical electrode, having a surface on which is arranged a first layer that comprises a platinum-iridium alloy, preferably consisting of a platinum-iridium alloy, and a second layer that comprises a conductive polymer, in particular an electrically conductive polymer, preferably consisting of a conductive polymer, in particular an electrically conductive polymer, and is arranged on the first layer,

characterized in that
the platinum-iridium alloy has an iridium content of at least 30 percent by weight based on the total mass of the platinum-iridium alloy.

In a preferred embodiment of the electrical conductor, the platinum-iridium alloy has an iridium content of at most 50 percent by weight based on the total mass of the platinum-iridium alloy. This preferred embodiment is a second embodiment of the invention, which is preferably dependent on the first embodiment of the invention.

In a preferred embodiment of the electrical conductor, the platinum-iridium alloy has an iridium content in a range of 30-50 percent by weight, preferably in a range of 30-45 percent by weight, more preferably in a range of 35-45 percent by weight, based on the total mass of the platinum-iridium alloy. This preferred embodiment is a third embodiment of the invention, which is preferably dependent on the first or second embodiment of the invention.

In a preferred embodiment of the electrical conductor, the platinum-iridium alloy is deposited galvanically, or in other words electrochemically, on the surface of the substrate. This preferred embodiment is a fourth embodiment of the invention, which is preferably dependent upon the first to third embodiments of the invention.

In a preferred embodiment of the electrical conductor, the platinum-iridium alloy has a surface, in particular a surface facing the second layer, with an average roughness value Rₐ in a range from 0.05 μm to 0.5 μm, preferably in a range from 0.1 μm to 0.4 μm, more preferably in a range from 0.1 μm to 0.3 μm, most preferably in a range from 0.2 μm to 0.3 μm. This preferred embodiment is a fifth embodiment of the invention, which is preferably dependent upon any one of the preceding embodiments of the invention.

In a preferred embodiment of the electrical conductor, the first layer, preferably the platinum-iridium alloy, has a layer thickness of at most 3 μm. This preferred embodiment is a sixth embodiment of the invention, which is preferably dependent upon any one of the preceding embodiments of the invention.

In a preferred embodiment of the electrical conductor, the first layer, preferably the platinum-iridium alloy, has a layer thickness of at least 300 nm. This preferred embodiment is a seventh embodiment of the invention, which is preferably dependent upon any one of the preceding embodiments of the invention.

In a preferred embodiment of the electrical conductor, the substrate comprises gold or platinum, preferably the substrate consists of gold or platinum. This preferred embodiment is an eighth embodiment of the invention, which is preferably dependent upon any one of the preceding embodiments of the invention.

In a preferred embodiment of the electrical conductor, the second layer comprises PEDOT, preferably Amplicoat®, preferably the second layer consists of PEDOT, preferably Amplicoat®. This preferred embodiment is a ninth embodiment of the invention, which is preferably dependent upon any one of the preceding embodiments of the invention.

A tenth embodiment of the invention is a method for manufacturing an electrical conductor according to any one of the preceding embodiments, comprising the following steps:
i) Provision of the substrate with the surface;
ii) Application of the first layer to the surface;
iii) Application of the second layer to the first layer.

In a preferred embodiment of the method, the first layer is applied to the surface by a galvanic, or in other words by an electrochemical, coating method. This preferred embodiment is an eleventh embodiment of the invention, which is preferably dependent upon the tenth embodiment of the invention.

In a preferred embodiment of the method, the second layer, in particular the conductive polymer, is applied to the first layer by electrochemical or chemical polymerization, by means of dip coating or by means of inkjet printing. This preferred embodiment is a twelfth embodiment of the invention, which is preferably dependent upon the tenth or eleventh embodiments of the invention.

### General

With respect to the embodiments described herein, the elements of which “have,” or “comprise,” a particular feature (for example, a material), in principle, a further embodiment is always contemplated in which the relevant element consists solely of the feature, i.e., does not comprise any other constituents.

The word “comprise” or “comprising” is used herein synonymously with the word “have” or “having.”

In one embodiment, if an element is denoted by the singular, an embodiment is also contemplated in which more than one such element is present.

The use of a term for an element in the plural in principle also encompasses an embodiment in which only a single corresponding element is included.

Unless otherwise indicated or clearly excluded from the context, it is possible in principle, and is hereby clearly contemplated, that features of different embodiments may also be present in the other embodiments described herein.

Likewise, it is contemplated in principle that all features described herein in connection with a method are also applicable to the products and devices described herein, and vice versa.

All such considered combinations are not explicitly listed in all instances, simply in order to keep the description brief.

Technical solutions known to be equivalent to the features described herein are also intended in principle to be encompassed by the scope of the invention.

In the present description, specifications of ranges also contain the values specified as limits. A specification of the type “in the range from X to Y” with respect to a quantity A consequently means that A can take the values X, Y and values between X and Y. One-sidedly limited ranges of the type “up to Y” for a size A accordingly mean as a value Y and less than Y.

Some of the features described are associated with the term “substantially.” The term “substantially” is to be understood in such a way that, under real conditions and manufacturing techniques, a mathematically exact interpretation of terms such as “superimposition,” “perpendicular,” “diameter” or “parallelism” can never be given exactly, but only within certain manufacturing error tolerances. For example, “substantially perpendicular axes” enclose an angle of 85 degrees to 95 degrees relative to one another, and “substantially equal volumes” comprise a variation of up to 5% by volume. For example, a “device consisting substantially of plastic” comprises a plastic content of ≥95 to ≤100% by weight. For example, a “substantially complete filling of a volume B” comprises a filling of ≥95 to ≤100% by volume of the total volume of B.

### DETAILED DESCRIPTION

A first object of the invention relates to an electrical conductor, in particular a medical electrode, having a surface on which is arranged a first layer that comprises a platinum-iridium alloy, preferably consisting of a platinum-iridium alloy, and a second layer that comprises a conductive polymer, preferably consisting of a conductive polymer, and is arranged on the first layer, characterized in that the platinum-iridium alloy has an iridium content of at least 30 percent by weight based on the total mass of the platinum-iridium alloy.

The electrical conductor is preferably a medical electrode for a medical device. Preferably, the electrical conductor has for this reason the shape of a line for a medical device. Examples of preferred shapes include cylindrical, annular, flat, spherical, needle-like, cubic, rectangular or a combination of two or more thereof. An annular shape, a flat shape or a cylindrical shape is particularly preferred. Most preferably, the electrical conductor is a ring electrode for a medical device or an electrode for a stent.

The electrical conductor can have the dimensions of a known electrode for a medical device. In one embodiment, it is preferred that the electrical conductor has a width in the range of 0.1 mm to 30.0 mm, more preferably in the range of 0.1 mm to 20.0 mm and further preferably in the range of 0.1 mm to 10.0 mm.

In a further embodiment, it is preferred that the electrical conductor has a diameter in the range of 0.1 mm to 30.0 mm, more preferably in the range of 0.1 mm to 20.0 mm and further preferably in the range of 0.1 mm to 10.0 mm.

In a further embodiment, it is preferred that the electrical conductor has a length in the range of 0.1 mm to 60.0 mm, more preferably in the range of 0.1 mm to 40.0 mm and further preferably in the range of 0.1 mm to 20.0 mm.

In a further embodiment, it is preferred that the electrical conductor has a height in the range of 0.1 mm to 30.0 mm, more preferably in the range of 0.1 mm to 20.0 mm and further preferably in the range of 0.1 mm to 10.0 mm.

A first layer is arranged on the surface, in particular the radially outer surface, of the substrate. Preferably, substantially the entire surface, in particular the radially outer surface, of the substrate is covered by the first layer.

The first layer comprises a platinum-iridium alloy. Preferably, the first layer consists substantially of the platinum-iridium alloy.

A second layer is arranged on the first layer, in particular radially on the outside of the first layer. Preferably, the first layer is substantially completely covered by the second layer.

The first layer is thus preferably present as an intermediate layer between the substrate and the second layer.

The second layer comprises a conductive, in particular electrically conductive, polymer. Preferably, the second layer consists substantially of the conductive polymer.

The platinum-iridium alloy of the first layer has an iridium content of at least 30 percent by weight based on the total mass of the platinum-iridium alloy. In other words, the platinum-iridium alloy consists of at least 30 percent iridium by weight. The remaining percent by weight of the platinum-iridium alloy is platinum and other optional constituents, wherein it is preferred that the platinum-iridium alloy consists substantially of platinum and iridium, wherein iridium occupies a content of at least 30 percent by weight and platinum occupies a content of at most 70 percent by weight.

An iridium content of at least 30 percent by weight based on the total mass of the platinum-iridium alloy has the surprising advantage that the second layer, in particular the conductive polymer of the second layer, has improved adhesion to the first layer. On the other hand, platinum-iridium alloys with lower iridium content have significantly lower adhesion of conductive polymers to their surface. Good adhesion is important to ensure the longest possible service life of the electrical conductor, in particular when used as a medical electrode, in particular when used as an implanted medical electrode.

In one embodiment of the electrical conductor, it is preferred that the platinum-iridium alloy has an iridium content of at most 50 percent by weight based on the total mass of the platinum-iridium alloy. In other words, the platinum-iridium alloy consists of a maximum of 50 percent iridium by weight. The remaining percent by weight of the platinum-iridium alloy is platinum and other optional constituents, wherein it is preferred that the platinum-iridium alloy consists substantially of platinum and iridium, wherein iridium occupies a content of at most 50 percent by weight and platinum occupies a content of at least 50 percent by weight.

An iridium content of at most 50 percent by weight based on the total mass of the platinum-iridium alloy has the surprising advantage that the platinum-iridium alloy has a comparatively high mechanical strength, in particular the platinum-iridium alloy has comparatively low brittleness. On the other hand, platinum-iridium alloys with a higher iridium content have a significantly reduced mechanical resilience, in particular a higher brittleness, such that the service life of the electrical conductor would be reduced, in particular when used as a medical electrode, in particular when used as an implanted medical electrode.

Furthermore, an iridium content higher than 50 percent by weight at the time of the invention would have a negative impact on the manufacturing cost of the electrical conductor due to the higher cost of iridium compared to platinum.

Preferably, the platinum-iridium alloy has an iridium content of at least 30 percent by weight and at most 50 percent by weight based on the total mass of the platinum-iridium alloy.

In one embodiment of the electrical conductor, the platinum-iridium alloy has an iridium content in a range of 30-50 percent by weight, preferably in a range of 30-45 percent by weight, more preferably in a range of 35-45 percent by weight, based on the total mass of the platinum-iridium alloy. These ranges represent a good compromise between improved adhesion of the second layer, in particular the conductive polymer of the second layer, to the first layer, in particular the platinum-iridium alloy of the first layer, and the mechanical strength of the first layer, in particular the platinum-iridium alloy of the first layer. Such iridium contents thus surprisingly provide an increased service life of the electrical conductor, in particular when used as a medical electrode, in particular when used as an implanted medical electrode.

The first layer can be applied to the substrate in different ways and thus, depending on the application method, have a different manifested surface in the direction of the second layer. Differently manifested surfaces in the direction of the second layer can have an influence on the adhesion of the second layer to the first layer.

For example, the first layer can be bonded, soldered, sintered, vapor-deposited or rolled onto the substrate.

In one embodiment of the electrical conductor, the first layer, in particular the platinum-iridium alloy, is deposited galvanically, i.e. electrochemically, on the surface of the substrate. One advantage here is that the substrate is only subjected to a low mechanical load when the first layer is applied. Furthermore, a galvanically applied platinum-iridium alloy can lead to improved adhesion of the second layer to the first layer due to its surface roughness, in particular its average roughness value Ra.

In one embodiment of the electrical conductor, the first layer, in particular the platinum-iridium alloy, has a surface with an average roughness value Ra in a range of 0.05-0.5 μm, preferably in a range of 0.1-0.4 μm, more preferably in a range of 0.1-0.3 μm, most preferably in a range of 0.2-0.3 μm. The aforementioned ranges refer to the average value of the average roughness value Ra. Preferably, the average roughness value Ra has a minimum value in a range of 0.05-0.2 μm and a maximum value in a range of 0.2-0.6 μm. Such ranges for the average roughness value Ra represent a good compromise between improved adhesion of the second layer to the first layer and mechanical stability of the first layer, in particular of the platinum-iridium alloy of the first layer. The higher the average roughness value Ra of the first layer, in particular of the platinum-iridium alloy, the greater the risk of detachment of same due to mechanical stress. For example, vertical elements of the first layer, in particular of the platinum-iridium alloy, protruding from the surface of the first layer could be detached from same by mechanical stress, for example friction. This would have a negative impact on the service life of the electrical conductor, in particular when used as a medical electrode, in particular when used as an implanted medical electrode.

In one embodiment of the electrical conductor, the first layer, in particular the platinum-iridium alloy, has a layer thickness of at most 3 μm, for example at most 2 μm or at most 1 μm. In some embodiments, the first layer has a layer thickness of at most 900, 800, 700, 600, or at most 500 nm.

In one embodiment of the electrical conductor, the first layer has a layer thickness of at least 300 nm, more preferably at least 400, 500, 600, 700, 800, 900 or 1000 nm. In some embodiments, the first layer has a layer thickness in a range of 500-1000 nm or in a range of 800-2000 nm.

The thickness of a layer can be determined by evaluating cross-sectional electron microscopic images, wherein the average distance between the profile lines along the opposing interfaces of a layer to be determined is calculated using suitable image processing software. More details on the determination of coating thickness by means of electron microscopy are described, for example, in Giurlani et al., Coatings 2020, 10, 1211; doi:10.3390/coatings10121211.

The substrate can comprise or consist of different materials as long as they are suitable for building up an electrical conductor, in particular for building up a medical electrode, in particular an implantable medical electrode.

In one embodiment of the electrical conductor, the substrate comprises a metal, a metal alloy or a combination thereof. A preferred metal is selected from the group consisting of iron, silver, copper, nickel, palladium, platinum, gold, iridium, titanium, hafnium, niobium, tantalum, cobalt, chromium, zirconium, rhenium, tungsten, molybdenum, and combinations, for example mixtures, preferably alloys, of at least two of such metals. A particularly preferred metal is gold or platinum. A particularly preferred alloy is steel. In one embodiment, it is preferred that the substrate comprises or consists of a platinum alloy, preferably a platinum-iridium alloy, such as PtIr10 or PtIr20.

The second layer can comprise or consist of different conductive polymers, in particular electrically conductive polymers. For example, the second layer can comprise or consist of one, two, three or more different conductive polymers.

A “conductive polymer” is understood to mean, in particular, an “electrically conductive polymer.”

In one embodiment, the conductive polymer, in particular the electrically conductive polymer, is cationic. In one embodiment, it is preferred that the conductive polymer comprises at least one or all of the following: a polyacetylene, a poly(vinyl alcohol), a poly(fluorene), a polyphenylene, a polyphenylenevinylene, a polypyrene, a polyazulene, a polynaphthalene, a poly(pyrrole), a polycarbazole, a polyindole, a polyazepine, a polyaniline, a polyacene, a polythiophene, a polythiophenevinylene, a poly(p-phenylene sulfide), a polypyridine or functionalized derivatives, precursors or mixtures thereof.

In one embodiment, it is preferred that the conductive polymer is derived from a functionalized derivative of EDOT selected from the group consisting of hydroxymethyl EDOT, EDOT vinyl, EDOT ether allyl, EDOTCOOH, EDOT MeOH, EDOT silane, EDOT vinyl, EDOT acrylate, EDOT sulfonate, EDOT amine, EDOT amide and combinations thereof. As an example, the functionalized derivative of 3,4-ethylenedioxythiophene (EDOT) can be selected from the group consisting of hydroxymethyl EDOT, EDOT vinyl, EDOT ether allyl, EDOT acrylate and combinations thereof.

In one embodiment, it is preferred that the second layer comprises or consists of PEDOT.

In one embodiment, it is preferred that the conductive polymer comprises an anionic photoreactive crosslinking agent. In this embodiment, it is preferred that the crosslinking agent has at least two photoreactive groups. In a further embodiment of the invention, it is preferred that the anionic photoreactive crosslinking agents comprise a compound of formula I: Xi˜Y˜X2, where Y is a radical containing at least one acidic group or salt of an acidic group, and Xi and X2 are each independently a radical containing a latent photoreactive group.

Examples of a photoreactive group is an aryl ketone or a quinone. In a further embodiment of the invention, it is preferred that spacers are part of Xi or X2, preferably together with the latent photoreactive group.

In one embodiment, it is preferred that, in the compound of formula I, Y is a radical that comprises at least one acidic group or salt thereof.

Examples of acidic groups include sulfonic acids, carboxylic acids, phosphonic acids and the like.

Examples of salts of such groups include sulfonate, carboxylate and phosphate salts. As an example, the crosslinking agent can include a sulfonic acid or sulfonate group.

In a further embodiment of the invention, it is preferred that such a photoreactive crosslinking agent is anionic.

Examples of counterions include alkali, alkaline earth metals, ammonium, protonated amines and the like.

In one embodiment, it is preferred that the conductive polymer comprises an anionic photoreactive hydrophilic polymer. In this embodiment, it is preferred that the hydrophilic polymer is anionic. Examples of anionic hydrophilic polymers include homopolymers, copolymers, terpolymers and the like. In a further embodiment of the invention, if the electrically conductive polymer comprises at least one anionic hydrophilic polymer, it is preferred that the anionic hydrophilic polymer is derivatized with photoreactive groups.

In a further embodiment, it is preferred that the anionic hydrophilic polymer comprises polymers comprising polyacrylamide and photoreactive groups (“photo-PA”). In a further embodiment of the invention, it is preferred that the anionic hydrophilic polymer comprises polyacrylamide and sulfonate groups. For example, the anionic hydrophilic polymer comprises acrylamido-2-methylpropane sulfonate (AMPS) groups and polyethylene glycol segments.

The terms “latent photoreactive group” and “photoreactive group” are used interchangeably and refer to a chemical entity that is sufficiently stable to remain in an inactive state (i.e., ground state) under normal storage conditions, but can undergo a transformation from the inactive state to an activated state when exposed to a suitable energy source. Unless otherwise specified, it is preferred that references herein to photoreactive groups include the reaction products of the photoreactive groups.

In a further embodiment, it is preferred that photoreactive groups are selected to respond to different proportions of actinic radiation.

For example, groups can be selected to be photoactivated using either ultraviolet or visible radiation.

Examples of photoreactive groups include azides, diazos, diazirines, ketones and quinones. In a further embodiment of the invention, it is preferred that the photoreactive group comprises an aryl ketone, such as acetophenone, benzophenone, anthrone, and anthrone-like heterocycles (i.e., heterocyclic analogs of anthrone, such as those with N, O, or S at the 10-position) or their substituted (for example, ring-substituted) derivatives. Examples of aryl ketones include heterocyclic derivatives of anthrone, including acridone, xanthone and thioxanthone and their ring-substituted derivatives. Other suitable photoreactive groups include quinone, such as anthraquinone.

Electrically conductive polymers are well known to a person skilled in the art and are commercially available under the trade names Orgacon®, available through Agfa-Gevaert N. V. (Belgium), or Amplicoat®, available through Heraeus Deutschland GmbH & Co. KG (Germany).

A further object of the invention relates to a method for manufacturing an electrical conductor, in particular a medical electrode, in particular an implantable medical electrode, according to any one of the preceding embodiments, comprising the following steps:
i) Provision of the substrate with the surface;
ii) Application of the first layer to the surface;
iii) Application of the second layer to the first layer.

The first layer can be applied by various methods. For example, the first layer can be applied by means of wet-chemical redox reactions. Preferably, the first layer, in particular the platinum-iridium alloy, can be applied to the substrate galvanically, i.e. by a galvanic coating method. This allows the layer thickness of the first layer to be set in a controlled and cost-effective manner. Furthermore, the surface roughness, in particular the average roughness value Ra of the surface of the first layer, in particular of the platinum-iridium alloy, can be set in a controlled manner. The selection of suitable process conditions and the correlation with the formed surface roughness, in particular the mean roughness Ra, is described in the technical literature in numerous publications. Suitable process conditions can be found, for example, in Petrossians et al., Journal of the Electrochemical Society, 158 (5), D269-D276 (2011).

In one embodiment of the method, the application of the second layer, in particular of the conductive polymer, to the first layer can be carried out, for example, by means of dip-coating or inkjet printing. In a further embodiment of the method, curing of the conductive polymer, if necessary, can be performed by means of chemical or electrochemical polymerization. The curing of PEDOT and its derivatives can preferably be effected electrochemically.

A further object of the invention relates to the use of a design of the electrical conductor described herein, in particular as a medical electrode, in particular as an implantable medical electrode.

A further object of the invention relates to the use of an embodiment of the method described herein for the manufacture of an electrical conductor.

In a further object of the invention, an electrical medical device having an electrical conductor according to any one of the preceding embodiments is provided.

The electrical medical device can be, for example, a lead for use with a pulse generator, pacemaker, cardiac resynchronization device, mapping catheter, sensor or stimulator.

Leads are electrical lines that can be used, for example, in medical technology applications such as cardiac stimulation, neuromodulation, deep brain stimulation, spinal cord stimulation or gastric stimulation.

In one embodiment, the lead is configured and/or intended to be connected to a generator of an active implantable device.

A lead can also be used in a medical device to pick up an electrical signal from the body of a living being.

A stimulator is a medical device that can produce a physiological effect by delivering an electrical signal to the body of a living being.

For example, by delivering an electrical signal to a nerve cell, a neurostimulator can create an electrical signal in the nerve cell (for example, an action potential).

A further embodiment relates to a microelectrode array that contains a plurality of electrical conductors according to the invention.

A further object of the invention relates to a diagnostic method in or on the body of a living being, comprising the recording of an electrical signal by means of the electrical conductor described herein.

A further object of the invention relates to the use of the electrical conductor described herein in a diagnostic method in or on the body of a living being, comprising the recording of an electrical signal by means of the electrical conductor.

A further object of the invention relates to a therapeutic method in or on the body of a living being, comprising the delivery of an electrical signal by means of the electrical conductor described herein.

A further object of the invention relates to the use of the electrical conductor described herein in a therapeutic method in or on the body of a living being, comprising the delivery of an electrical signal by means of the electrical conductor.

The therapeutic method may involve the delivery of an electrical signal to nerve cells or muscle cells in the region of an organ, for example, heart, muscle, stomach or brain.

The diagnostic method can involve the capture of an electrical signal from nerve cells or muscle cells in the region of an organ, for example, heart, muscle or brain.

A further object of the invention relates to an electrode that comprises at least one electrical conductor according to the invention. In this case, it is preferred that the electrode is suitable for use in an implantable medical device and more preferably in an active implantable medical device (AIMD). In one embodiment, the electrode according to the present invention is suitable for use in a temporary or short-term medical device such as a catheter. In one embodiment, the electrode is suitable for use in a line for a medical device.

The features disclosed for the electrical conductor are also disclosed for the method for manufacturing the electrical conductor and all other objects of the invention, and vice versa.

### Description of the Method

To determine the average roughness value Ra, 3D images were taken using the psurf custom confocal microscope (NanoFocus AG, Germany) on the surface of the corresponding electrical conductor in substrate form, which images comprised the reference section S. By means of the software pSoft Analysis Premium (7.4.8872; NanoFocus AG, Germany), the microscopic 3D images were analyzed. For this purpose, any deflection of the substrate in the 3D images was initially corrected (use of a polynomial of degree 2). The reference sections S (for example, with a width of 0.0315 mm) were then defined and the roughness profile along the reference section S was obtained using a Gaussian filter (0.08 mm). In each case, at least three reference sections S running parallel to one another of the same length were selected. In this case, the reference sections S had a length of at least 20% of the circumference of the substrate. From the roughness profiles obtained along the reference sections S, the average roughness value Ra was determined according to the version of the standard DIN EN ISO 4287 valid at the filing date.

A swipe test was carried out to determine the mechanical load capacity of a layer of a conductive polymer applied to surfaces. For this purpose, the electrical conductors were provided in substrate form (diameter at least 5 cm) and immersed in a phosphate-buffered saline (PBS) solution for 15 min at room temperature.

The substrates were then fastened using an adhesive tape (Scotch Magic Tape) one after the other to a sample table that could be moved in the Y direction. Above the sample table in the initial position and directly above the substrate was a holder for receiving a rod, wherein the rod was mounted so that it could move freely in the vertical direction and be placed on the substrate. A foam (10×10 mm area) soaked in PBS solution was fastened to the end of the rod pointing at the substrate, before the rod was carefully placed on the substrate fastened to the sample table. A starting weight of 43 g was applied to the substrate by the rod mounted in this way. The sample table was then moved back and forth 10 times in the Y direction, such that the rod is displaced over the substrate with its weight force being applied to the substrate in each case. After the substrate had been detached and dried, it was photographed at a suitable magnification on a microscope under normal light.

The entire process was then repeated, wherein the rod was weighted by incrementally increasing the weight by 10 g each time (by attaching two washers, each weighing 5 g). The process was preferably repeated until detachment of the conductive polymer was visible. Since the electrical polymer used was Amplicoat®, available from Heraeus Deutschland GmbH & Co. KG, which appears black under normal light, the detachment was clearly visible.

It will be apparent to a person skilled in the art that, instead of the swipe test described herein, other equivalent means can be used in a similar manner to determine the mechanical strength of surfaces.

### EXAMPLES

The invention is further illustrated below using examples which are, however, not to be understood as limiting. It will be apparent to a person skilled in the art that other equivalent means may be used similarly in place of the features described here.

The following applies to comparative examples 1 and 2 and to examples 1 and 2 according to the invention. An electrical conductor in the form of a disk was provided. The disk has a diameter of 5 cm and a height of 0.1 cm and has a substantially smooth surface, which has not been etched or coated in any way by any other method.

The following applies to comparative example 2 and to example 2 according to the invention. The disk was galvanically coated with a layer of platinum-iridium alloy. These coatings have a thickness of approximately 2 μm and an average roughness value Ra of approximately 0.2 μm.

Finally, all disks were coated with a conductive polymer according to the manufacturer's instructions (Amplicoat®, available from Heraeus Deutschland GmbH & Co. KG, Hanau, Germany). In comparative example 1 along with example 1 according to the invention, the conductive polymer was applied directly to the surface of the disks. In comparative example 2 and example 2 according to the invention, the conductive polymer was applied to the platinum-iridium alloy layer.

The mechanical load capacity of the applied layer of the conductive polymer was then determined by means of the swipe test. In this case, a higher weight value prior to a detachment of the conductive polymer correlates with a higher mechanical load capacity.

Additional details for each of the respective examples are provided below and in Table 1.

### Comparative Example 1

The disk consists of a platinum-iridium alloy with an iridium content of 10 percent by weight based on the total mass of the platinum-iridium alloy (PtIr10), and there is no additional intermediate layer between the surface of the disk and the conductive polymer. This example thus simulates an electrical conductor having a first layer of such a low iridium platinum-iridium alloy, wherein the first layer is formed substantially smooth.

### Comparative Example 2

As in comparative example 1, the disk consists of a platinum-iridium alloy having an iridium content of 10 percent by weight based on the total mass of the platinum-iridium alloy (PtIr10), wherein the galvanically deposited intermediate layer described above is formed between the disk and the conductive polymer. The intermediate layer, like the disk, consists of a PtIr10 alloy. This example thus simulates an electrical conductor with a galvanically deposited first layer of PtIr10 with an average roughness value Ra of 0.2 μm.

### Example 1 According to the Invention

This example is similar to comparative example 1, wherein the disk consists of a platinum-iridium alloy with an iridium content of 40 percent by weight based on the total mass of the platinum-iridium alloy (PtIr40). This example thus simulates an electrical conductor having a first layer of a PtIr40 alloy according to the invention, wherein the first layer is formed substantially smooth.

### Example 2 According to the Invention

As in example 1 according to the invention, the disk consists of a PtIr10 alloy, wherein the galvanically deposited intermediate layer described above is formed between the disk and the conductive polymer. The intermediate layer consists of a PtIr40 alloy. This example thus simulates an electrical conductor with a first layer of a PtIr40 alloy according to the invention, wherein the first layer is galvanically deposited and has an average roughness value of 0.2 μm.

TABLE 1
Determination of the mechanical load capacity of the
conductive polymer layer on different coatings.

| | | | | Example 1 | Example 2 | |
|---|---|---|---|---|---|---|
| | Compar- | Compar- | | according | according | |
| | ative | ative | | to the | to the | |
| | example 1 | example 2 | | invention | invention | |
| Material of the | PtIr10 | PtIr10 | | PtIr40 | PtIr40 | |
| simulated first layer | | | | | | |
| Galvanically deposited | no | yes | | no | yes | |
| first layer | | | | | | |
| Average roughness | Substan- | 0.2 | μm | Substan- | 0.2 | μm |
| value Rₐ of the | tially | | | tially | | |
| simulated first layer | smooth | | | smooth | | |
| Weight at which | 93 g | 143 | g | 133 g | >203 | g |
| conductive polymer | | | | | | |
| layer is delaminated | | | | | | |

From the direct comparison of comparative example 1 to example 1 according to the invention and from the direct comparison of comparative example 2 to example 2 according to the invention, it can be seen that the improvement in the mechanical stability of the electrical conductor is primarily due to the high iridium content of the platinum-iridium alloy and only secondarily to the surface roughness of the layer to which the conductive polymer was applied. The first layer of the examples according to the invention, in particular the galvanically deposited first layer of example 2 according to the invention, thus allows the construction of more durable, mechanically stable electrical conductors, in particular medical electrodes.

### FIGURES

The invention is further illustrated by way of example below by means of figures. The invention is not limited to the figures.

The following are shown

FIG. 1 a cross-sectional electron micrograph of an electronic conductor according to the invention,

FIG. 2 an electron micrograph of a galvanically deposited first layer in a top view,

FIG. 3 test results of a swipe test of example 2 according to the invention in a top view,

FIG. 4 test results of a swipe test of comparative example 1 in a top view, and

FIG. 5 a flow chart of a method for manufacturing an electrical conductor.

### DESCRIPTION OF THE FIGURES

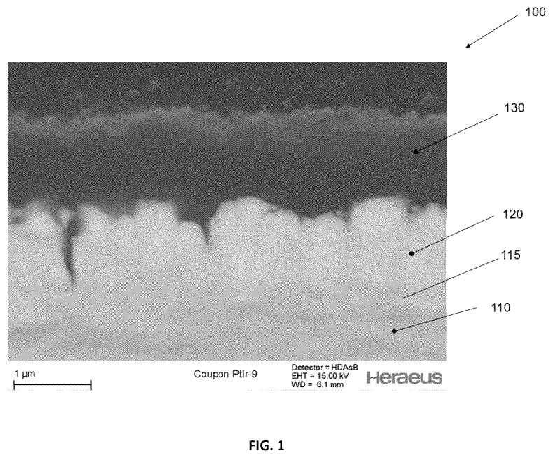

FIG. 1 shows a cross-sectional electron micrograph of an electrical conductor 100 according to the invention. The electrical conductor 100 comprises a platinum-iridium alloy substrate 110 having an iridium content of 10 percent by weight based on the total mass of the platinum-iridium alloy (PtIr10) with a surface 115. A platinum-iridium alloy having an iridium content of 40 percent by weight based on the total mass of the platinum-iridium alloy (PtIr40) was galvanically applied to the surface 115 of the substrate 110 as a first layer 120. In the embodiment shown, the first layer 120 consists of the platinum-iridium alloy having the iridium content of 40 percent by weight based on the total mass of the platinum-iridium alloy. The first layer 120 has a layer thickness between 0.8 μm and 1.2 μm. On the side of the first layer 120 facing away from the substrate 110, a conductive polymer in the form of Amplicoat®, available from Heraeus Deutschland GmbH & Co. KG, is applied as a second layer 130. In the embodiment shown, the second layer 130 consists of the conductive polymer. The second layer 130 has a layer thickness between 0.8 μm to 1.5 μm.

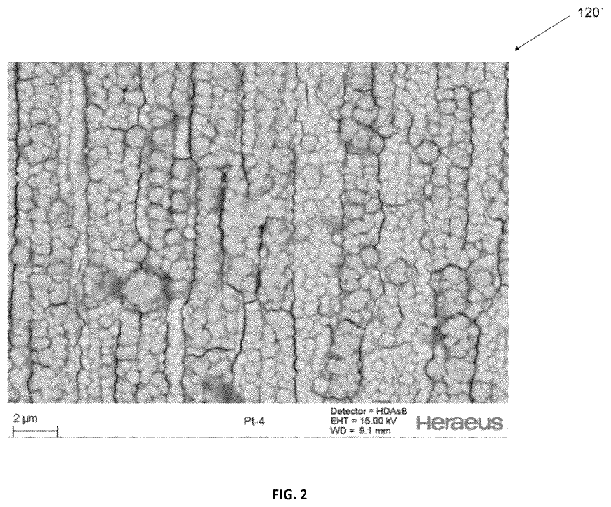

FIG. 2 shows an electron micrograph of a first layer 120′ according to the invention in a top view. The second layer 120′ consists of a platinum-iridium alloy with an iridium content of 40 percent by weight based on the total mass of the platinum-iridium alloy (PtIr40) and was galvanically applied to a substrate (not visible) and has a surface with elevations and depressions. The average roughness value Ra of the first layer 120′ shown is 0.254 μm.

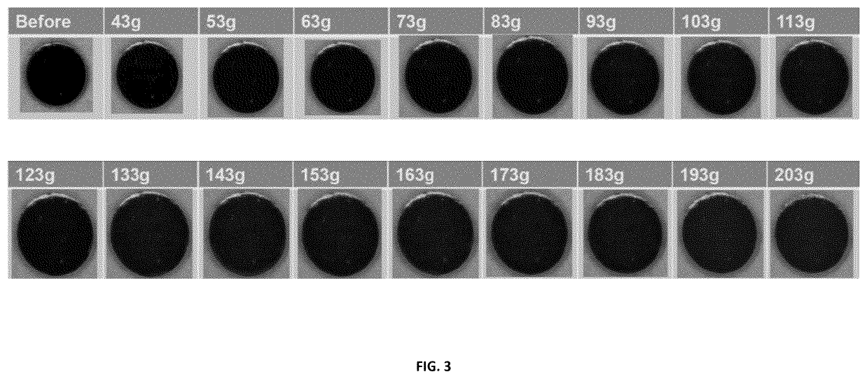

FIG. 3 shows the results of the swipe test of example 2 according to the invention (see Table 1). It can be clearly seen that up to a weight value of 203 g, no detachment of the conductive polymer (black) has taken place.

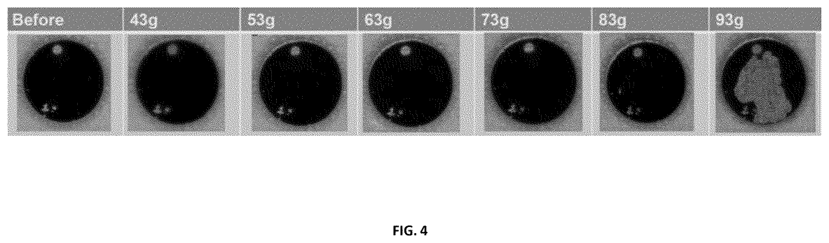

FIG. 4 shows the results of the swipe test of comparative example 1 (see Table 1). Up to a weight value of 83 g, there is no detachment of the electrical polymer (black). At a weight value of 93 g, a part of the electrical polymer has been rubbed off and the first layer (silver-colored) under the electrical polymer can be seen.

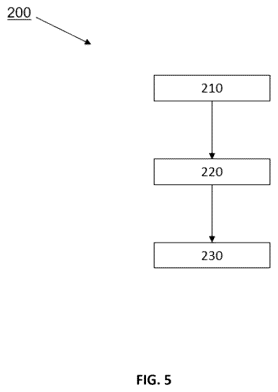

FIG. 5 shows a flow chart of a method 200 for manufacturing an electrical conductor 100 according to the invention comprising the steps 210 to 230.

In a step 210, a substrate 110 having a surface 115 is provided.

In a step 220, a first layer 120 is applied to the surface 115 of the substrate 110. For example, in step 220, the application is performed by a galvanic coating method.

In a step 230, a second layer 130 is applied to the first layer 120, in particular to the side of the first layer 120 facing away from the substrate 110. The application of the second layer 130 to the first layer 120 can be accomplished, for example, by electrochemical or chemical polymerization, by means of dip-coating or by means of inkjet printing or a combination thereof.

### REFERENCE SIGNS

100 Electrical conductor
110 Substrate
115 Surface of substrate
120, 120′ First layer
130 Second layer
200 Method
210 Provision
220 Application of first layer
230 Application of second layer

1. An electrical conductor comprising a substrate having a surface on which is arranged a first layer that comprises a platinum-iridium alloy and a second layer that comprises a conductive polymer and is arranged on the first layer, wherein
the platinum-iridium alloy has an iridium content of at least 30 percent by weight based on the total mass of the platinum-iridium alloy.

2. The electrical conductor according to claim 1, wherein the platinum-iridium alloy has an iridium content of at most 50 percent by weight based on the total mass of the platinum-iridium alloy.

3. The electrical conductor according to claim 1, wherein the platinum-iridium alloy has an iridium content in a range of 30-50 percent by weight, preferably in a range of 30-45 percent by weight, more preferably in a range of 35-45 percent by weight, based on the total mass of the platinum-iridium alloy.

4. The electrical conductor according to claim 1, wherein the platinum-iridium alloy is galvanically deposited on the surface of the substrate.

5. The electrical conductor according to claim 1, wherein the platinum-iridium alloy has a surface with an average roughness value Rₐ in a range of 0.05 μm to 0.5 μm.

6. The electrical conductor according to claim 1, wherein the first layer has a layer thickness of at most 3 μm.

7. The electrical conductor according to claim 6, wherein the first layer has a layer thickness of at least 300 nm.

8. The electrical conductor according to claim 1, wherein the substrate comprises or consists of gold or platinum.

9. The electrical conductor according to claim 1, wherein the second layer comprises or consists of PEDOT.

10. A method for manufacturing an electrical conductor according to claim 1, comprising the following steps:
i) Provision of the substrate with the surface;
ii) Application of the first layer to the surface;
iii) Application of the second layer to the first layer.

11. The method according to claim 10, wherein the first layer is applied to the surface by a galvanic coating method.

12. The method according to claim 10, wherein the application of the second layer to the first layer is performed by electrochemical or chemical polymerization, by means of dip-coating or by means of inkjet printing.
